(19) United States
(12) Patent Application Publication
(10) Pub. No.: US 2010/0113897 A1
(43) Pub. Date: May 6, 2010

(54) CONTINUOUS ANALYTE MONITORING ASSEMBLY AND METHODS OF USING THE SAME

(75) Inventors: Allen J. Brenneman, Goshen, IN (US); James E. Smous, Niles, MI (US)

Correspondence Address: NIXON PEABODY LLP, 300 S. Riverside Plaza, 16th Floor, CHICAGO, IL, 60606-6613, US

(73) Assignee: Bayer Health Care LLC, Tarrytown, NY (US)

(21) Appl. No.: 12/530,157

(22) PCT Filed: Mar. 14, 2008
(86) PCT No.: PCT/US08/03375

§ 371 Date: Sep. 4, 2009

Related U.S. Application Data
(60) Provisional application No. 60/918,813, filed on Mar. 19, 2007.

(51) Int. Cl. A61B 5/1459; A61B 5/145; A61B 5/1473; B23P 11/00

(52) U.S. Cl. 600/310; 600/309; 600/345; 29/428; 600/365

(57) ABSTRACT

A continuous analyte monitoring assembly is adapted to assist in determining an analyte level of a fluid. The monitoring assembly includes a housing, electronics, an implantable sensor and a cannula. The housing has a bottom in which the bottom forms a recess. The electronics are located within the housing and assist in determining an analyte level of a fluid sample. The sensor moves from a retracted position to an inserted position using the recess. The cannula assists in placing the implantable sensor.

### CROSS-REFERENCE TO RELATED APPLICATION

This application is a nationalized application of PCT/US2008/003375 filed on Mar. 14, 2008, which claims the benefit of priority of U.S. Provisional Application No. 60/918,813, filed on Mar. 19, 2007, which both are incorporated by reference in their entireties.

### FIELD OF THE INVENTION

The present invention relates generally to a continuous analyte monitoring assembly and methods of using the same. More particularly, the invention relates to a continuous analyte monitoring assembly that includes an implantable sensor that is adapted to be placed in the body to assist in determining the analyte level (e.g., a concentration) of a fluid (e.g., blood).

### BACKGROUND OF THE INVENTION

The quantitative determination of analytes in body fluids is of great importance in the diagnoses and maintenance of certain physiological abnormalities. For example, lactate, cholesterol, and bilirubin should be monitored in certain individuals. In particular, determining glucose in body fluids is important to diabetic individuals who must frequently check the glucose level in their body fluids to regulate the glucose intake in their diets. The results of such tests may be used to determine what, if any, insulin or other medication needs to be administered.

The analyte may be continuously monitored using a sensor. One of the challenges of using a continuous analyte monitoring process is to minimize the number of acts to (a) attach the sensor/electronics to the skin and (b) insert the implantable sensor that assists in determining the analyte level of a fluid (e.g., blood). It would be desirable to minimize such acts in a continuous analyte monitoring process. Additionally, it would be desirable to make the continuous analyte monitoring assembly as small as possible to reduce any interference with normal activities of a user. It would also be desirable to control location (especially depth) of the sensor in the skin.

It would be desirable to have a continuous analyte monitoring assembly and methods of using the same that provide one or more of these desirable benefits.

### SUMMARY OF THE INVENTION

In one embodiment, a continuous analyte monitoring assembly is adapted to assist in determining an analyte level of a fluid. The continuous analyte monitoring assembly comprises a housing, electronics, an implantable sensor and a cannula. The housing has a bottom in which the bottom forms a recess. The electronics are located within the housing and the electronics are adapted to assist in determining an analyte level of a fluid sample. The implantable sensor is adapted to move from a retracted position to an inserted position. The implantable sensor is adapted to move through the recess. The cannula is adapted to assist in the placement of the implantable sensor.

According to one method, a continuous analyte monitoring assembly is formed. Housing is provided having a bottom in which the bottom forms a recess. Electronics are located within the housing. The electronics are adapted to assist in determining an analyte level of a fluid sample. An implantable sensor is located at least partially within the housing. The implantable sensor is adapted to move from a retracted position to an inserted position. At least a portion of the implantable sensor is adapted to move through the recess to the inserted position. A cannula is provided that is adapted to assist in the placement of the implantable sensor. The cannula is adapted to move between a retracted position and an inserted position.

According to another method, a method of using a continuous analyte monitoring assembly is performed. A continuous analyte monitoring assembly is provided. The continuous analyte monitoring assembly includes housing, electronics, an implantable sensor, and a cannula. The electronics are adapted to assist in determining an analyte level of a fluid sample. The cannula is adapted to assist in placing of the implantable sensor. The continuous analyte monitoring assembly is attached to the skin. The implantable sensor is inserted into the skin from a retracted position to an inserted position by rotating a portion of the continuous analyte monitoring assembly with respect to the remainder of the continuous analyte monitoring assembly.

### BRIEF DESCRIPTION OF THE DRAWINGS

FIG. 1 is a top perspective view of a continuous analyte monitoring assembly according to one embodiment.

FIG. 2 is a cut-away view of FIG. 1.

FIG. 3 is an exploded view of the continuous analyte monitoring assembly of FIG. 1.

FIG. 4a is an exploded view of a disposable assembly of the continuous analyte monitoring assembly of FIG. 1.

FIG. 4b is a top perspective view of an assembled disposable assembly of FIG. 4a.

FIG. 5a is an exploded view of a reusable assembly of the continuous analyte monitoring assembly of FIG. 1.

FIG. 5b is a side cutaway view of the assembled reusable assembly of FIG. 5a.

FIG. 6a is a bottom perspective view of the continuous analyte monitoring assembly of FIG. 1 in a retracted position.

FIG. 6b is an enlarged bottom perspective view of a cannula according to one embodiment in the continuous analyte monitoring assembly of FIG. 1 in a retracted position.

FIG. 7 is a side view of the continuous analyte monitoring assembly of FIG. 1 in an inserted position.

FIG. 8 is a partial cutaway bottom perspective view of an inserter according to one embodiment with a loaded continuous analyte monitoring assembly of FIG. 1.

FIG. 9 is a continuous analyte monitoring assembly according to one embodiment.

FIG. 10 is a continuous analyte monitoring assembly in communication with a receiving module according to one embodiment.

### DESCRIPTION OF ILLUSTRATED EMBODIMENTS

The present invention relates generally to a continuous analyte monitoring assembly and methods of using the same. More particularly, the continuous analyte monitoring assembly uses an implantable sensor that is adapted to be placed in the body to assist in determining the analyte level of a fluid (e.g., blood). The implantable sensor is adapted to be placed in the subcutaneous region of the skin. It is contemplated that the sensor may be placed in other regions of the skin such as the dermis. The implantable sensor is adapted to be placed into the body for up to 3 days in one method. In another method, the implantable sensor is adapted to be placed into the body for up to 5 or 7 days. It is contemplated that the implantable sensor may be placed in the body for even longer time periods.

The implantable sensor assists in determining the “level” of the desired analyte. The term “level” is defined herein as including any information related to, for example, the amount, relative concentration and absolute concentration. The term “level” as defined herein also includes changes in the amount, relative and absolute concentrations, whether in a percentage or absolute context. These “level” changes may be used over a selected duration of time such as, for example, a time change in amount or concentration. The “level” may refer to a time change in amount or concentration, and compared to a later time change.

Analytes that may be measured include glucose, lipid profiles (e.g., cholesterol, triglycerides, LDL, and HDL), fructose, lactate, and/or bilirubin. Analytes could also include therapeutic drugs, metabolites of the therapeutic drug or other substances that are affected by the therapeutic drug being analyzed. It is contemplated that other analyte levels may be determined. One non-limiting example of the sensor's use is to determine the glucose concentration of the fluid. The fluid may be an intercellular fluid or an intracellular fluid. These fluids include interstitial fluid (ISF), blood, plasma and other fluids.

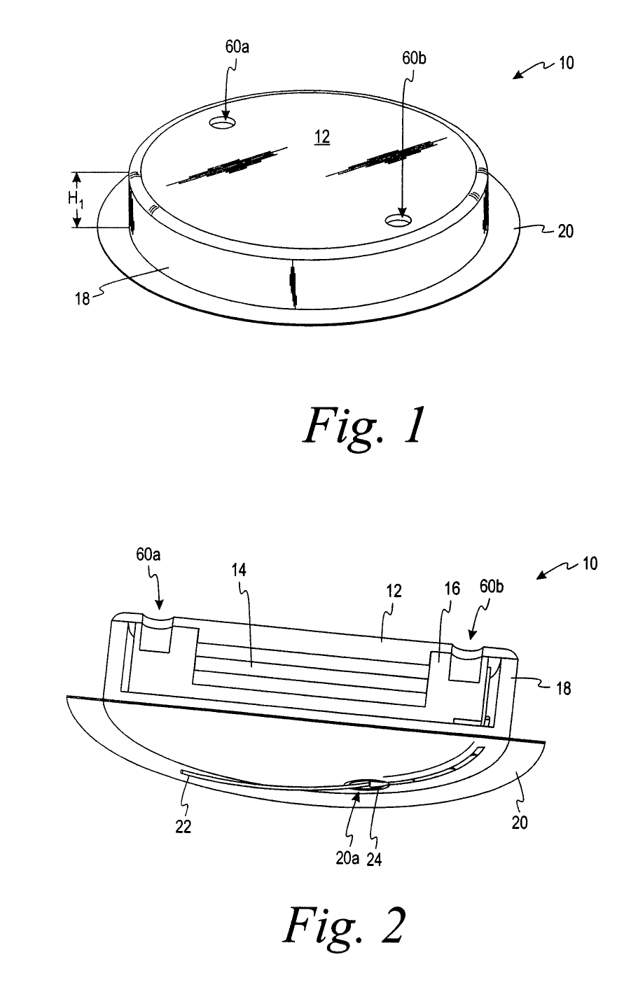

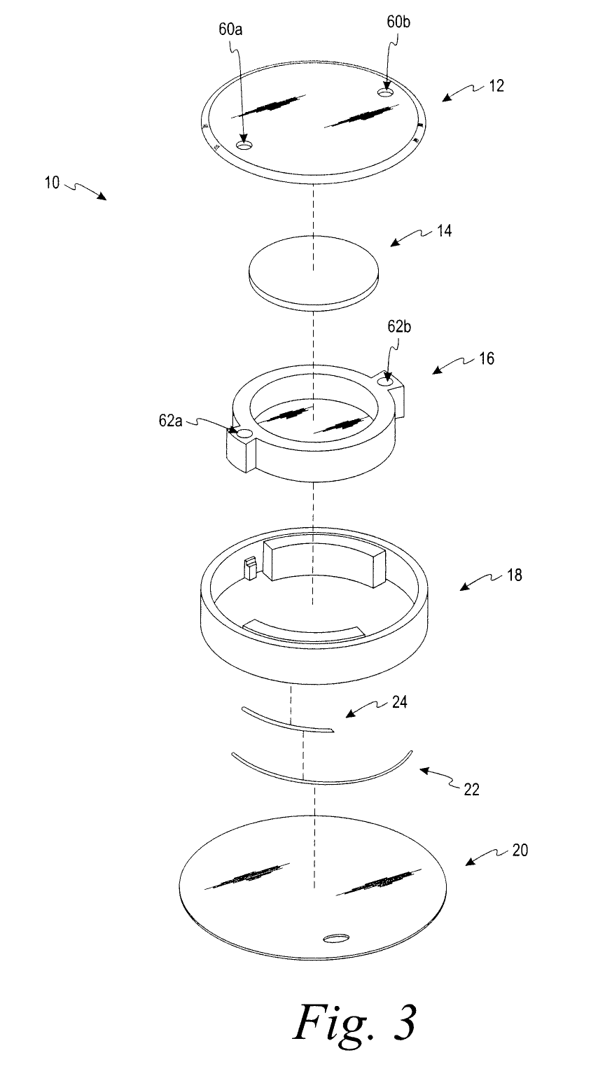

Referring to FIGS. 1-3, a continuous analyte monitoring assembly 10 is shown according to one embodiment. The continuous analyte monitoring assembly 10 includes a cover 12, electronics mounted on a printed circuit board 14, printed circuit board housing 16, disposable housing 18, an adhesive liner 20, an implantable sensor 22, a cannula 24 and a connector 26 (FIG. 3). The continuous analyte monitoring assembly 10 of FIG. 1 is shown as being generally circular. It is contemplated that the continuous analyte monitoring assembly may be of other shapes.

The continuous analyte monitoring assembly may further include a battery. The battery is typically located on the printed circuit board. The battery may be a rechargeable battery such as an inductively rechargeable battery. The battery may be recharged by being loaded into a docking station.

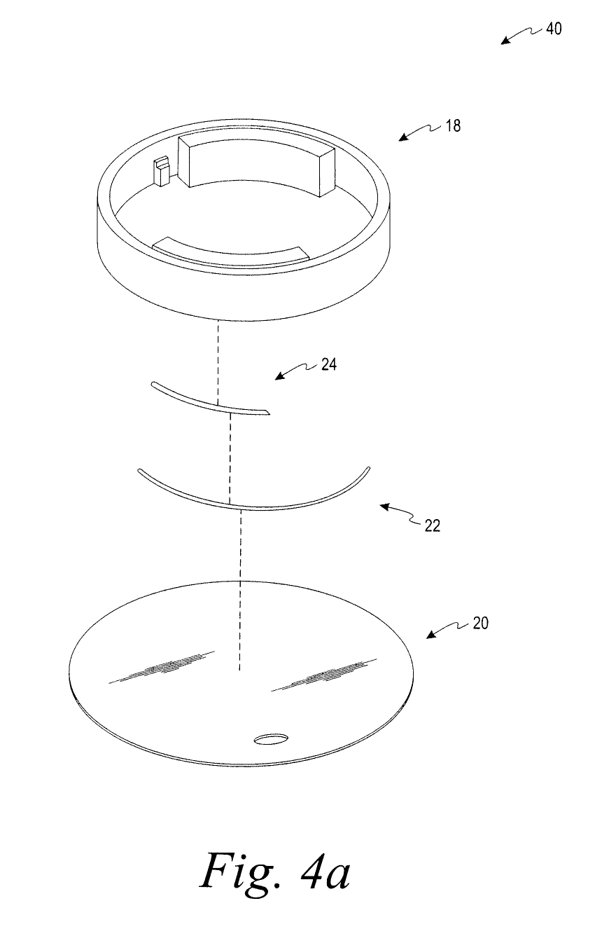

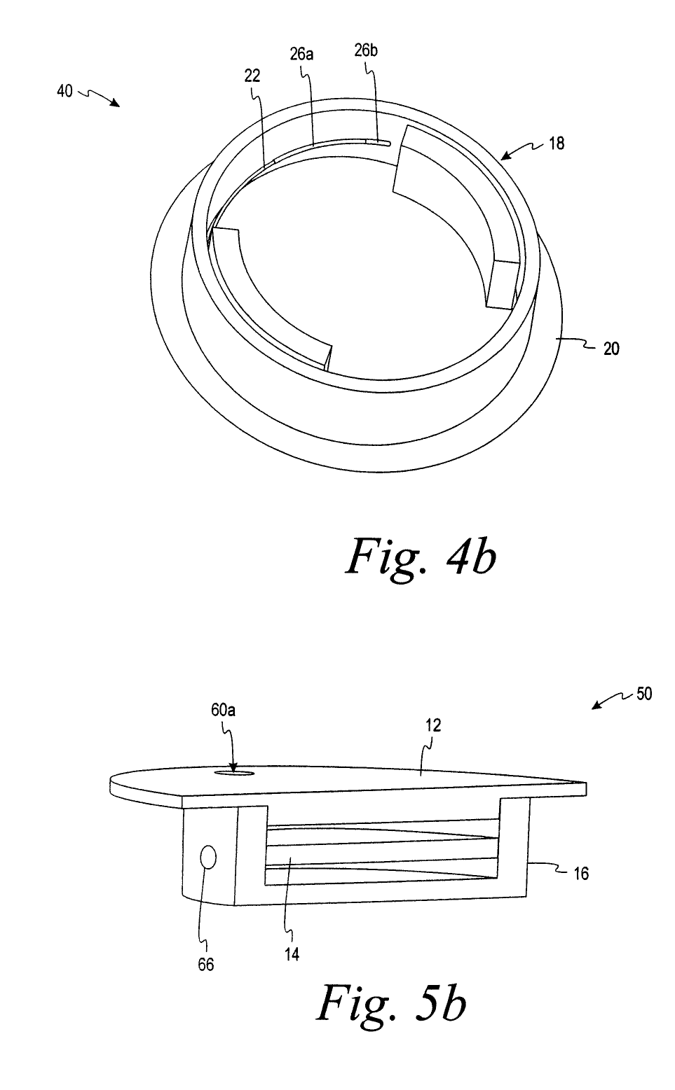

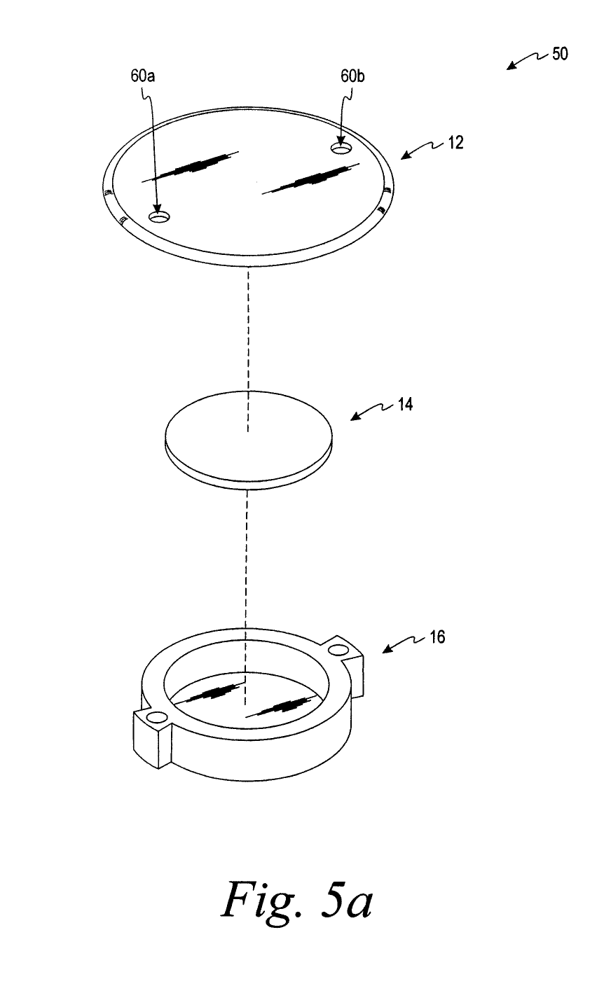

The continuous analyte monitoring assembly may be a disposable assembly, a reusable assembly, or a combination thereof. For example, the continuous analyte monitoring assembly 10 includes a disposable assembly 40 (see FIGS. 4a, 4b) and a reusable assembly 50 (see FIGS. 5a, 5b). In this embodiment, the disposable assembly 40 of FIGS. 4a, 4b includes the disposable housing 18, the adhesive liner 20, the implantable sensor 22, the cannula 24 and the connector 26. As will be discussed below, the cannula 24 assists in locating the sensor 22 in the skin. The connector 26 assists in mechanically and electrically connecting the implantable sensor 22 and the electronics (e.g., printed circuit board 14). The connector may include an anode portion 26a and a cathode portion 26b. It is contemplated that other connections may be used to connect the sensor 22 and the printed circuit board 14 of the continuous analyte monitoring assembly 10. For example, a connector may have a plurality of contact points (e.g., 3-6 contact points) that assist in mechanically and electrically connecting the implantable sensor and the printed circuit board. The connector may include contacts points such as a reference portion or a temperature-monitoring component.

The reusable assembly 50 of FIGS. 5a, 5b includes the cover 12, the printed circuit board 14 and the printed circuit board housing 16. When the reusable assembly 50 is placed into the disposable assembly 40, the implantable sensor 22 is mechanically and electrically connected to the electronics mounted on the printed circuit board 14 in the reusable assembly 50. The connector 26 of the disposable assembly 40 extends through an aperture 66 formed in the printed circuit board housing 16 to mechanically and electrically connect the sensor 22 and the printed circuit board 14. To connect the reusable assembly 50 and the disposable assembly 40 in one method, the reusable assembly 50 is inserted and rotated into the disposable assembly 40. It is contemplated that the reusable assembly and the disposable assembly may be connected together by other methods. In one method, the reusable assembly and the disposable assembly are rotated and snapped into each other.

Referring back to FIGS. 1-3, the cover 12 assists in protecting the remainder of the continuous analyte monitoring assembly 10. Specifically, the cover 12 assists in preventing or inhibiting contaminants from reaching the electronics mounted on the printed circuit board 14. Non-limiting examples of contaminants include moisture, liquid or particles such as dust. It is contemplated that the cover 12 may assist in preventing or inhibiting other contaminants from reaching the printed circuit board 14. The cover 12 also forms a plurality of apertures 60a, 60b to assist in coupling or connecting with an inserter that will be discussed below in detail with respect to FIG. 8. It is contemplated that the cover may include a different number or different shaped apertures to assist in connecting with an inserter. It is also contemplated that the cover may include other features to assist in coupling the continuous analyte monitoring assembly and an inserter.

The printed circuit board housing 16 also assists in preventing or inhibiting contaminants from reaching the printed circuit board 14 such as those contaminants previously discussed. The printed circuit board housing 16 assists in maintaining the location of the printed circuit board 14. The printed circuit board housing 16 also assists in electrically insulating the printed circuit board 14.

The cover 12 and the printed circuit board housing 16 may be made from a variety of materials. One example of a material that may be used in forming the cover and the printed circuit board housing is polymeric material. Non-limiting examples of polymeric materials that may be used to form the cover and the printed circuit board housing include ABS, polycarbonate and acrylic.

The printed circuit board 14 contains the electronics that are used in the continuous analyte monitoring assembly 10. It is contemplated that the electronic components used in the continuous analyte monitoring assembly 10 may be located on a device other than a printed circuit board. The electronic components in one embodiment include three subassemblies—a power supply, a potentiostat and a communications link. In one embodiment, the electronic components assist in operating the implantable sensor, monitoring the signals from the sensor, converting analog signals to digital signals, and storing data from the sensor. The printed circuit board 14 assists in processing the information by directing and collecting data for the analysis. The printed circuit board may be a potentiostat if an electrochemical analysis is to be performed. The continuous analyte monitoring assembly 10 may include two modules in which a first module is used to collect/analyze the data with a second module being used to transmit or communicate the data. It is contemplated that the electronic components may assist in performing additional functions in the continuous analyte monitoring assembly 10.

The disposable housing 18 assists in preventing or inhibiting contaminants such as those previously discussed from reaching the sensor 22 and also from reaching the cannula 24 and the connector 26. The disposable housing 18 also assists in holding components such as the connector 26 and the cannula 24 in specific locations therein. The disposable housing 18 is attached to the adhesive liner 20.

The adhesive liner 20 includes a detachable liner and an adhesive thereon that assists in attaching the continuous analyte monitoring assembly 10 to the skin. The detachable liner is adapted to be peeled off from the remainder of the continuous analyte monitoring assembly before the continuous analyte monitoring assembly is attached to the skin. The adhesive liner 20 forms an aperture 20a that allows the sensor 22 to extend therethrough.

In one embodiment, the implantable sensor 22 is a wire. The wire typically includes electrically conductive material with a portion of the wire containing reagent that is adapted to react with the desired analyte. The electrically conductive material is typically a noble metal such as platinum or platinum-iridium. It is contemplated that other electrically conductive materials (metal or non-metal) may be used in forming the implantable sensor. The wire is typically coated with an insulating material to assist in (a) protecting the electrically conductive material from corrosive effects and (b) providing an insulating effect on the electrically conductive material. The insulting material is typically a polymeric material. Non-limiting polymeric materials include polyimides, polytetrafluoroethylene (TEFLON®), ultraviolet-curable polymers, heat-curable polymers, and natural rubber. In one example, a reagent (e.g., glucose oxidase enzyme) may be included with the wire if the desired analyte to be tested in glucose.

The implantable sensor is typically an electrochemical sensor. It is contemplated, however, that the implantable sensor may be an optical sensor. If the implantable sensor was an optical sensor in one embodiment, a fiber optic may be coated to react with the analyte such that a fluorescence, reflectance or absorption change would be created.

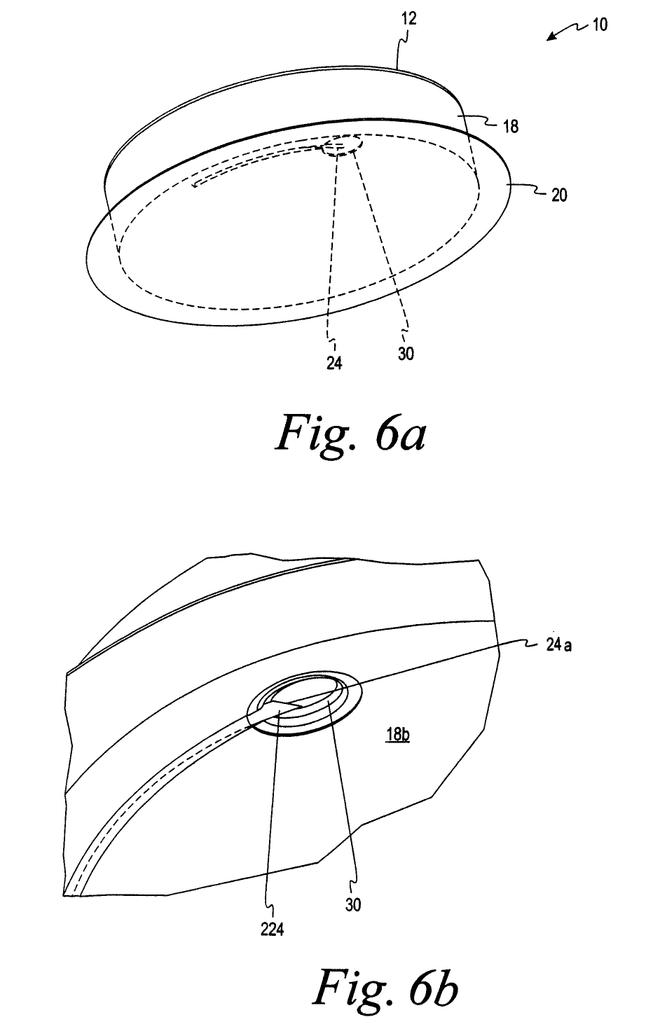

One non-limiting example of the cannula 24 is best shown in FIGS. 6a, 6b. FIG. 6b is an enlarged view of a portion of the cannula 24 with the adhesive liner 20 having been removed. The cannula 24 has a sharpened end 24a that assists in inserting the implantable sensor 22 into the body. The cannula 24 is shown as extending into a recess 30 formed in a bottom 18a of the disposable housing 18. The recess 30 assists in controlling the depth of the placement of the implantable sensor 22. Specifically, during the process of inserting the implantable sensor 22, the skin extrudes or raises up into the recess 30 when sufficient pressure is applied, resulting in a more consistent location of the implantable sensor 22. The recess 30 also assists in securing the skin contact with the cannula 24. The recess may be formed in different shapes and sizes from that depicted in FIGS. 6a, 6b. It is generally desirable, however, for the recess to be generally wide and shallow to assist in (a) controlling the depth of the placement and (b) extruding or raising up of the skin.

During the insertion act, the cannula and the implantable sensor are moved together, but during the retraction act only the cannula is moved, which results in the placement of the implantable sensor in the body. To move the cannula between retracted and insertion positions, the continuous analyte monitoring assembly may include two torsion springs in which one torsion spring drives the cannula in the insertion act and the other torsion spring moves the cannula in the retraction act. It is contemplated that the cannula may be moved by other mechanisms. In another method, another mechanical mechanism may be used in combination with a spring. For example, a mechanical mechanism such as cam-like drive system may be used in combination with a retraction spring.

When using a cannula in a continuous analyte monitoring assembly, the continuous analyte monitoring assembly needs to be sized to accommodate the length of the implantable sensor plus the length of the cannula in the retracted position. The continuous analyte monitoring assembly using a rotary motion to insert the implantable sensor can be smaller than a continuous analyte monitoring assembly using a linear insertion motion because in one embodiment the sensor and the cannula are curved and fit in the circumference of the rotary-motion, continuous analyte monitoring assembly.

Referring back to FIG. 1, in a generally circular embodiment, the diameter of the continuous analyte monitoring assembly is generally less than about 1 inch. More specifically, the diameter of the continuous analyte monitoring assembly is generally less than about 0.8 inch. The height (H₁) of the continuous analyte monitoring assembly is generally less than about 0.5 inch. More specifically, the height of the continuous analyte monitoring assembly is generally less than about 0.33 or about 0.2 inch. The area of the continuous analyte monitoring assembly is generally less than about 0.5 in². More specifically, the area of the continuous analyte monitoring assembly is generally less than about 0.25 or about 0.2 in². To reduce interference with normal activities of the user, it is desirable to form a continuous analyte monitoring assembly that is small in size. More specifically, the larger the continuous analyte monitoring assembly 10 (especially in its height—see H₁ of FIG. 1) the easier it is to dislodge or detach from the body or skin.

It is desirable to minimize the number of acts involved in attaching the continuous analyte monitoring assembly 10 to the skin and inserting the implantable sensor 22 in the body. This is especially important if the user is inserting the implantable sensor at home without any assistance.

In one method, the continuous analyte monitoring assembly 10 includes the following acts: (a) loading a continuous analyte monitoring assembly with implantable sensor into an inserter, (b) removing the adhesive liner from the remainder of the continuous analyte monitoring assembly; and (c) placing the inserter with the continuous analyte monitoring assembly on the body. In one method, the inserter is pressed down onto the skin. In another method, the inserter may be pressed down and rotated onto the skin. It is contemplated that the continuous analyte monitoring assembly may be attached to the skin by using methods other than an adhesive. The attachment methods may be chemical, mechanical or a combination thereof. In this method, it is not necessary to attach the electronics as a separate act because the electronics are already in communication with the sensor.

Referring to FIG. 8, an inserter (inserter 110) is shown according to one embodiment. FIG. 8 shows the inserter 110 being partially cut away to better depict the coupling of the continuous monitoring analyte assembly 10 and the inserter 110. The inserter is designed to be reusable. It is contemplated, however, that the inserter may be disposable. The inserter 110 of FIG. 8 is shown with a loaded continuous analyte monitoring assembly 10 therein and, more specifically, the inserter 110 is shown as being placed over the top of the cover 12 of the continuous analyte monitoring assembly 10. The inserter 110 forms an opening or cavity that is adapted to receive the continuous analyte monitoring assembly 10. In this opening, the inserter 110 includes a plurality of projections or pins 112a, 112b that extend generally downward. The pins 112a, 112b correspond with the apertures 60a, 60b formed in the cover 12 and the apertures 62a, 62b of the printed circuit board housing 16 (see FIG. 3). Thus, the pins 112a, 112b extend through the apertures 60a,b, 62a,b and desirably form a snug fit therein to couple the continuous analyte monitoring assembly 10 and the inserter 110.

In one method, the continuous analyte monitoring assembly 10 is applied to the skin using an adhesive. The adhesive liner 20 is then removed from the remainder of the continuous analyte monitoring assembly 10. It is contemplated that the adhesive liner 20 may be removed from the remainder of the continuous analyte monitoring assembly 10 prior to being loaded into the inserter 110. This, however, is generally not desirable because the adhesive is prematurely exposed, which may lead to adhesive getting in unwanted locations. One non-limiting example of an adhesive is a cyanoacrylate. It is contemplated that other attachment methods may be used such as a chemical attachment, a mechanical attachment or a combination thereof. One mechanical attachment method is an arm or leg band. Another type of mechanical attachment method uses a vacuum or other pressure to attach the continuous analyte monitoring assembly 10.

In this method, the inserter 110 may then be cocked by rotating, for example, a plunger 114 a quarter turn and then back. The cocking of the inserter 110 may be performed before or after the inserter 110 with the loaded continuous analyte monitoring assembly 10 is pressed against the skin. In this method, the inserter and the continuous analyte monitoring assembly 10 are pressed hard to reach a predetermined force such that the plunger 114 is pressed down and the sensor 22 is driven under the skin. The design of the continuous analyte monitoring assembly assists in facilitating a simple rotary motion to insert the sensor in the body. The inserter 110 is typically spring-loaded such the reusable assembly 40 (including the cover 12 and the printed circuit board housing 16) are rotated at a sufficient force to penetrate and drive the sensor 22 into the skin. In this embodiment, the spring force pressure must be overcome before the plunger is depressed. The predetermined force may be set by a spring rate and designed at a pressure where the skin extrudes into the recess 30. One example of a spring that may be used is a torsion spring. It is contemplated that other types of springs may be used to rotate and drive the sensor into the skin.

To assist in preventing or inhibiting accidental insertion of the sensor 22, a safety button 116 may be included on the inserter that would need to be activated before the plunger 114 could be moved. It is also contemplated that other mechanisms may be used to rotate and drive the sensor into the skin. For example, a small release button may be used instead of a plunger.

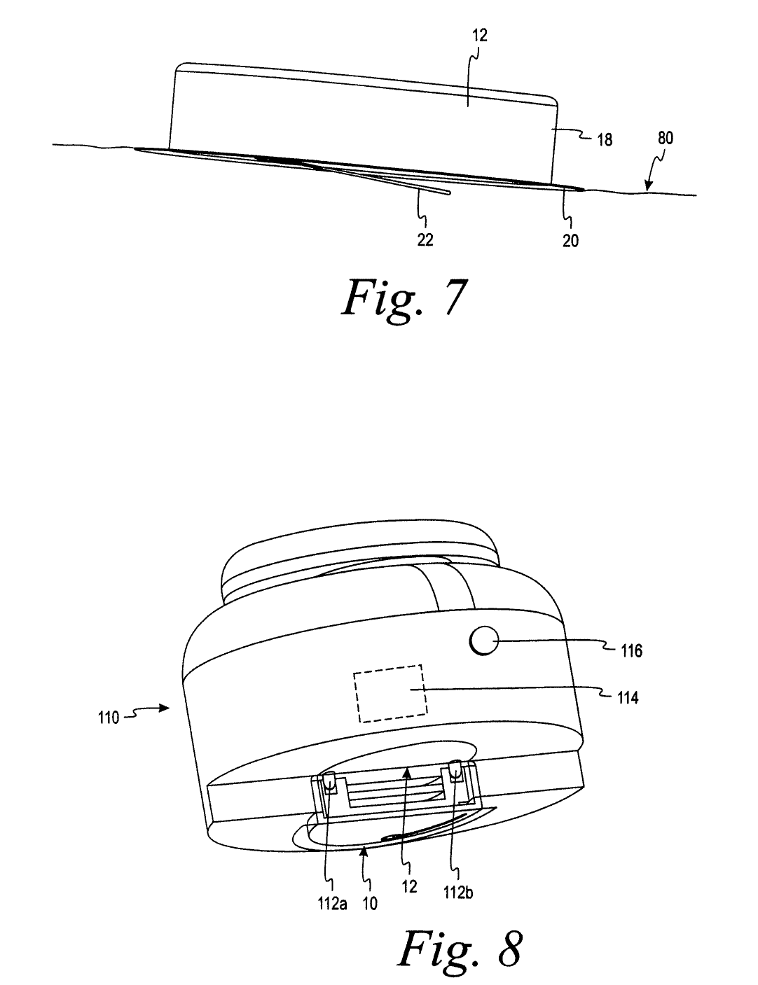

When using the cannula 24, during the rotation of the continuous analyte monitoring assembly 10, the disposable housing 18 is stationary. The remainder of the components of the continuous analyte monitoring assembly 10 are rotated with respect to the disposable housing 18. The rotation may be done in the counterclockwise direction in one method. In another method, the rotation may be done in the clockwise direction. The continuous analyte monitoring assembly 10 is shown in FIG. 7 as being in the retracted position on skin 80.

The rotary motion of the continuous analyte monitoring assembly 10 is desirable because it results in a smaller footprint for the continuous analyte monitoring assembly. This results in a smaller footprint because the implantable sensor may be curved to fit in the circumference of the continuous analyte monitoring assembly. In such an embodiment, the cannula may also be curved to fit in the circumference of the continuous analyte monitoring assembly. Additionally, the rotary motion is desirable because an angle is used with the insertion of the sensor, which results in improved depth control in the locating of the implantable sensor.

After the implantable sensor 22 has been driven under the skin, the cannula 24 is retracted. The inserter 110 is then removed from the continuous analyte monitoring assembly and is ready to be reused if desired.

In one embodiment, the continuous analyte monitoring assembly is connected to a remote-monitoring system over a communications link. The communications link between the continuous analyte monitoring assembly and the remote-monitoring system may be wireless, hard wired or a combination thereof. The wireless communications link may include an RF link, an infrared link or an inductive magnetic link. The wireless implementation may include an interne connection. The continuous analyte monitoring assembly may communicate via its communication interface with devices such as a computer, e-mail server, cell phone or telephone. It is contemplated that the continuous analyte monitoring assembly may include other devices that are adapted to store, send and/or receive information.

The remote-monitoring system enables an individual such as a physician to monitor the analyte. The remote-monitoring system may be located in, for example, a hospital. The physician may be able to access information from the continuous analyte monitoring assembly via its communications interface using, for example, a computer or telephone. The remote-monitoring system is especially desirable for patients who are less lucid and need assistance with monitoring the analyte. It is desirable for the remote-monitoring system to be able to display, calibrate and store information received from the continuous analyte monitoring assembly.

The remote-monitoring system may be used to send back instructional information to the patients. In such an embodiment, a continuous analyte monitoring assembly includes a communications link that has a receiver component to receive instructions from the remote-monitoring system in addition to a transmitter component to transmit information to the remote-monitoring system.

In one method, the continuous analyte monitoring assembly may forward information over a communications link in real-time. In another method, the continuous analyte monitoring assembly may store and process the data before forwarding the information over a communications link in another embodiment.

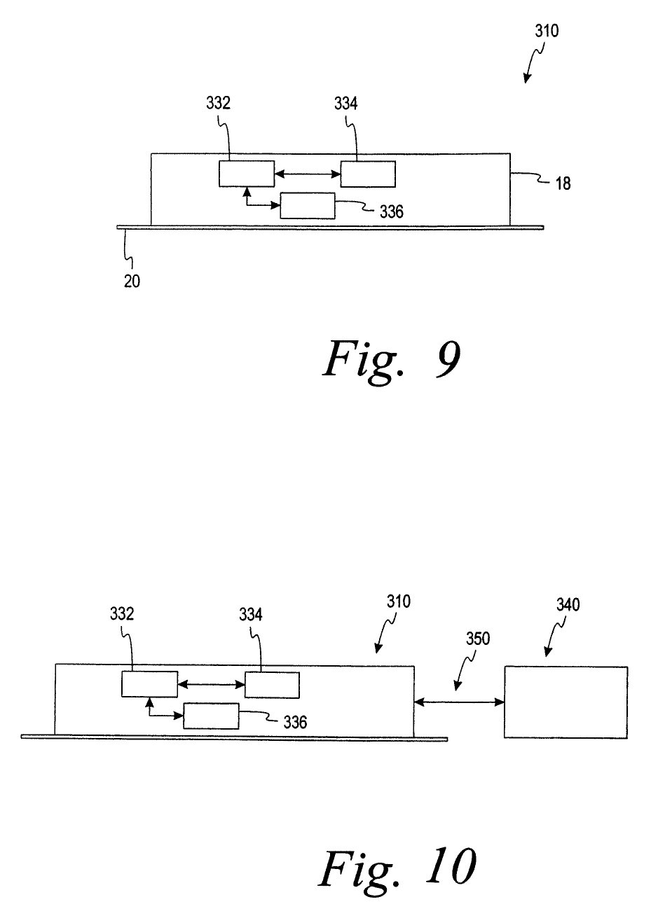

Referring to FIG. 9, a continuous analyte monitoring assembly 310 includes a processor 332, memory 334 and a communication interface 336. It is contemplated that the continuous analyte monitoring assembly 10 may include a processor, memory and a communications interface as described above in monitoring assembly 310. Referring to FIG. 10, the continuous analyte monitoring assembly 310 is shown in communication with a receiving module 340 (e.g., a remote-monitoring station) over a communications link 350.

### Alternative Embodiment A

A continuous analyte monitoring assembly adapted to assist in determining an analyte level of a fluid, the continuous analyte monitoring assembly comprising:

a housing having a bottom, the bottom forming a recess;

electronics located within the housing, the electronics being adapted to assist in determining an analyte level of a fluid sample;

an implantable sensor being adapted to move from a retracted position to an inserted position, the implantable sensor being adapted to move through the recess; and

a cannula being adapted to assist in placing the implantable sensor.

### Alternative Embodiment B

The assembly of Alternative Embodiment A, wherein the electronics is a printed circuit board.

### Alternative Embodiment C

The assembly of Alternative Embodiment A, further including a removable adhesive liner, the adhesive liner being adapted to attach to skin.

### Alternative Embodiment D

The assembly of Alternative Embodiment A, wherein the housing includes disposable housing and reusable housing, the reusable housing being adapted to contain the electronics.

### Alternative Embodiment E

The assembly of Alternative Embodiment D, wherein the disposable housing includes the implantable sensor, the cannula and an adhesive liner.

### Alternative Embodiment F

The assembly of Alternative Embodiment A, wherein the implantable sensor is a wire that includes a reagent.

### Alternative Embodiment G

The assembly of Alternative Embodiment A, wherein the implantable sensor is an electrochemical sensor.

### Alternative Embodiment H

The assembly of Alternative Embodiment A, wherein the implantable sensor is an optical sensor.

### Alternative Embodiment I

The assembly of Alternative Embodiment A, wherein the area of the continuous analyte monitoring assembly is less than 0.25 in².

### Alternative Embodiment J

The assembly of Alternative Embodiment A, wherein the height of the continuous analyte monitoring assembly is less than 0.5 in.

### Alternative Embodiment K

The assembly of Alternative Embodiment A, wherein the level of the analyte is a concentration of the analyte.

### Alternative Process L

A method of forming a continuous analyte monitoring assembly, the method comprising the acts of:

providing a housing having a bottom, the bottom forming a recess;

locating electronics within the housing, the electronics being adapted to assist in determining an analyte level of a fluid sample;

locating an implantable sensor at least partially within the housing, the implantable sensor being adapted to move from a retracted position to an inserted position, at least a portion of the implantable sensor being adapted to move through the recess to the inserted position; and

providing a cannula being adapted to assist in the placement of the implantable sensor, the cannula being adapted to move between a retracted position and an inserted position.

### Alternative Process M

The method of Alternative Process L, further including attaching a removable adhesive liner to the housing.

### Alternative Process N

The method of Alternative Process L, wherein the housing includes disposable housing and reusable housing, the reusable housing being adapted to contain the electronics.

### Alternative Process O

The method of Alternative Process N, wherein the disposable housing includes the implantable sensor, the cannula and an adhesive liner.

### Alternative Process P

The method of Alternative Process L, wherein the implantable sensor is a wire that includes a reagent.

### Alternative Process Q

The method of Alternative Process L, wherein the implantable sensor is an electrochemical sensor.

### Alternative Process R

The method of Alternative Process L, wherein the implantable sensor is an optical sensor.

### Alternative Process S

A method of using a continuous analyte monitoring assembly, the method comprising the acts of:

providing a continuous analyte monitoring assembly, the continuous analyte monitoring assembly including housing, electronics, an implantable sensor, and a cannula, the electronics being adapted to assist in determining an analyte level of a fluid sample, the cannula being adapted to assist in placing the implantable sensor;

attaching the continuous analyte monitoring assembly to the skin; and

inserting the implantable sensor into the skin from a retracted position to an inserted position by rotating a portion of the continuous analyte monitoring assembly with respect to the remainder of the continuous analyte monitoring assembly.

### Alternative Process T

The method of Alternative Process S, wherein the continuous analyte monitoring assembly further includes a removable adhesive liner, the removable adhesive liner being attached to the housing.

### Alternative Process U

The method of Alternative Process S, wherein the housing includes disposable housing and reusable housing, the reusable housing being adapted to contain the electronics.

### Alternative Process V

The method of Alternative Process U, wherein the disposable housing includes the implantable sensor, the cannula and an adhesive liner.

### Alternative Process W

The method of Alternative Process S, wherein the implantable sensor is a wire that includes a reagent.

### Alternative Process X

The method of Alternative Process S, wherein the implantable sensor is an electrochemical sensor.

### Alternative Process Y

The method of Alternative Process S, wherein the implantable sensor is an optical sensor.

### Alternative Process Z

The method of Alternative Process S, wherein the continuous analyte monitoring assembly is attached to the skin using an adhesive.

### Alternative Process AA

The method of Alternative Process S, wherein the continuous analyte monitoring assembly is attached to the skin using a mechanical method.

### Alternative Process BB

The method of Alternative Process S, wherein the analyte is glucose.

### Alternative Process CC

The method of Alternative Process S, wherein the analyte is a therapeutic drug, a metabolite of a therapeutic drug or a substance that is affected by a therapeutic drug.

### Alternative Process DD

The method of Alternative Process S, wherein the implantable sensor is inserted into the skin using an inserter, the inserter forming an opening or cavity that is adapted to assist in receiving and securing the continuous analyte monitoring assembly.

### Alternative Process EE

The method of Alternative Process S, further including the continuous analyte monitoring assembly communicating with a receiving module.

While the invention is susceptible to various modifications and alternative forms, specific embodiments and methods thereof have been shown by way of example in the drawings and are described in detail herein. It should be understood, however, that it is not intended to limit the invention to the particular forms or methods disclosed, but, to the contrary, the intention is to cover all modifications, equivalents and alternatives falling within the spirit and scope of the invention as defined by the appended claims.

1. A continuous analyte monitoring assembly adapted to assist in determining an analyte level of a fluid, the continuous analyte monitoring assembly comprising:
a housing having a bottom, the bottom forming a recess;
electronics located within the housing, the electronics being adapted to assist in determining an analyte level of a fluid sample;
an implantable sensor being adapted to move from a retracted position to an inserted position, the implantable sensor being adapted to move through the recess; and
a cannula being adapted to assist in placing the implantable sensor.

2. The assembly of claim 1, wherein the electronics is a printed circuit board.

3. The assembly of claim 1, further including a removable adhesive liner, the adhesive liner being adapted to attach to skin.

4. The assembly of claim 1, wherein the housing includes disposable housing and reusable housing, the reusable housing being adapted to contain the electronics.

5. The assembly of claim 4, wherein the disposable housing includes the implantable sensor, the cannula and an adhesive liner.

6. The assembly of claim 1, wherein the implantable sensor is a wire that includes a reagent.

7. The assembly of claim 1, wherein the implantable sensor is an electrochemical sensor.

8. The assembly of claim 1, wherein the implantable sensor is an optical sensor.

9. The assembly of claim 1, wherein the area of the continuous analyte monitoring assembly is less than 0.25 in².

10. The assembly of claim 1, wherein the height of the continuous analyte monitoring assembly is less than 0.5 in.

11. (canceled)

12. A method of forming a continuous analyte monitoring assembly, the method comprising the acts of:
providing a housing having a bottom, the bottom forming a recess;
locating electronics within the housing, the electronics being adapted to assist in determining an analyte level of a fluid sample;
locating an implantable sensor at least partially within the housing, the implantable sensor being adapted to move from a retracted position to an inserted position, at least a portion of the implantable sensor being adapted to move through the recess to the inserted position; and
providing a cannula being adapted to assist in the placement of the implantable sensor, the cannula being adapted to move between a retracted position and an inserted position.

13. The method of claim 12, further including attaching a removable adhesive liner to the housing.

14. (canceled)

15. The method of claim 12, wherein the housing includes disposable housing, the disposable housing includes the implantable sensor, the cannula and an adhesive liner.

16. The method of claim 12, wherein the implantable sensor is a wire that includes a reagent.

17. The method of claim 12, wherein the implantable sensor is an electrochemical sensor.

18. The method of claim 12, wherein the implantable sensor is an optical sensor.

19. A method of using a continuous analyte monitoring assembly, the method comprising the acts of:
providing a continuous analyte monitoring assembly, the continuous analyte monitoring assembly including housing, electronics, an implantable sensor, and a cannula, the electronics being adapted to assist in determining an analyte level of a fluid sample, the cannula being adapted to assist in placing the implantable sensor;
attaching the continuous analyte monitoring assembly to the skin; and
inserting the implantable sensor into the skin from a retracted position to an inserted position by rotating a portion of the continuous analyte monitoring assembly with respect to the remainder of the continuous analyte monitoring assembly.

20. The method of claim 19, wherein the continuous analyte monitoring assembly further includes a removable adhesive liner, the removable adhesive liner being attached to the housing.

21. (canceled)

22. The method of claim 21, wherein the disposable housing includes the implantable sensor, the cannula and an adhesive liner.

23. The method of claim 19, wherein the implantable sensor is a wire that includes a reagent.

24-27. (canceled)

28. The method of claim 19, wherein the analyte is glucose.

29. The method of claim 19, wherein the analyte is a therapeutic drug, a metabolite of a therapeutic drug or a substance that is affected by a therapeutic drug.

30. The method of claim 19, wherein the implantable sensor is inserted into the skin using an inserter, the inserter forming an opening or cavity that is adapted to assist in receiving and securing the continuous analyte monitoring assembly.

31. The method of claim 19, further including the continuous analyte monitoring assembly communicating with a receiving module.
